SEQUENCE LISTING (20 sequences)

SEQ ID NO: 1 - protein, 448 aa, synthetic construct
  REGION 1..448: note = Light-chain of Botulinum Toxin A type
MPFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNK

SEQ ID NO: 2 - DNA, 27 bp, synthetic construct
  misc_feature 1..27: note = Forward primer for BT-LC K89R
  source 1..27: mol_type = other DNA
gataattatttaaggggagttacaaaa

SEQ ID NO: 3 - DNA, 27 bp, synthetic construct
  misc_feature 1..27: note = Reverse primer for BT-LC K89R
  source 1..27: mol_type = other DNA
ttttgtaactccccttaaataattatc

SEQ ID NO: 4 - DNA, 24 bp, synthetic construct
  misc_feature 1..24: note = Forward primer for BT-LC K212R
  source 1..24: mol_type = other DNA
ggtgcaggcagatttgctacagat

SEQ ID NO: 5 - DNA, 24 bp, synthetic construct
  misc_feature 1..24: note = Reverse primer for BT-LC K212R
  source 1..24: mol_type = other DNA
atctgtagcaaatctgcctgcacc

SEQ ID NO: 6 - DNA, 27 bp, synthetic construct
  misc_feature 1..27: note = Forward primer for BT-LC K301R
  source 1..27: mol_type = other DNA
cttaataaagctagatcaatagtaggt

SEQ ID NO: 7 - DNA, 27 bp, synthetic construct
  misc_feature 1..27: note = Reverse primer for BT-LC K301R
  source 1..27: mol_type = other DNA
acctactattgatctagctttattaag

SEQ ID NO: 8 - DNA, 24 bp, synthetic construct
  misc_feature 1..24: note = Forward primer for BT-LC K330R
  source 1..24: mol_type = other DNA
acatctggaagattttcggtagat

SEQ ID NO: 9 - DNA, 24 bp, synthetic construct
  misc_feature 1..24: note = Reverse primer for BT-LC K330R
  source 1..24: mol_type = other DNA
atctaccgaaaatcttccagatgt

SEQ ID NO: 10 - DNA, 27 bp, synthetic construct
  misc_feature 1..27: note = Forward primer for BT-LC K335R
  source 1..27: mol_type = other DNA
ttttcggtagatagattaaaatttgat

SEQ ID NO: 11 - DNA, 27 bp, synthetic construct
  misc_feature 1..27: note = Reverse primer for BT-LC K335R
  source 1..27: mol_type = other DNA
atcaaattttaatctatctaccgaaaa

SEQ ID NO: 12 - protein, 448 aa, synthetic construct
  REGION 1..448: note = BT-LC (K89R)
MPFVNKQFNYKDPVNGVDIAYIKIPNVGQMQPVKAFKIHNKIWVIPERDTFTNPEEGDLNPPPEAKQVPVSYYDSTYLSTDNEKDNYLRGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNK

SEQ ID NO: 13 - protein, 448 aa, synthetic construct
  REGION 1..448: note = BT-LC (K212R)
MPFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGRFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNK

SEQ ID NO: 14 - protein, 448 aa, synthetic construct
  REGION 1..448: note = BT-LC (K301R)
MPFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKARSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNK

SEQ ID NO: 15 - protein, 448 aa, synthetic construct
  REGION 1..448: note = BT-LC (K330R)
MPFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGRFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNK

SEQ ID NO: 16 - protein, 448 aa, synthetic construct
  REGION 1..448: note = BT-LC (K335R)
MPFVNKQFNYKDPVNGVDIAYIKIPNVGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDRLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNK

SEQ ID NO: 17 - protein, 448 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNK

SEQ ID NO: 18 - DNA, 1347 bp, synthetic construct
  source 1..1347: mol_type = other DNA
atgccctttgtcaacaaacagttcaactacaaggacccagttaatggagtagacatcgcatatatcaagattcccaacgctggccagatgcaacccgttaaggcatttaaaatccataacaaaatctgggttatcccagagcgggataccttcaccaaccccgaggagggcgatctgaaccccccgccggaggcgaagcaggtcccagtgagctactacgatagcacctacctcagcaccgacaacgagaaggacaactacctcaaaggagtcacgaagttgttcgagagaatctactccacagacctcggccgcatgcttctaaccagcattgtgcgtggcattcccttttggggcggctctaccatcgacacagagctgaaggtgatagacaccaactgcatcaacgtaatccagcctgacggcagctaccgaagcgaggagcttaacctggtgatcatcggcccttccgccgatatcatccaattcgagtgcaagagcttcggccacgaggtcctgaacctcacccggaacggctatggaagcacccagtacataagattcagccctgacttcaccttcgggtttgaggagagcttggaggtcgacacaaaccccctgctgggagccgggaggttcgccactgacccagccgtgactctggcacacgagctgatccacgccggtcaccgcctgtacggcatagctataaacccaaacagggtgttcaaagtgaacaccaacgcttactatgaaatgagcggcctggaggtgagcttcgaggagctgagaacgttcgggggacatgatgctaaatttatcgacagcctgcaggagaacgagttcaggctgtactactacaataagttcaaggatatagcgagcactctgaacaaggccaagtccatcgtaggcactactgcatccctccagtatatgaagaatgtgttcaaagagaaatacctgctgagcgaggataccagcggtaagttcagcgtggataagcttaagttcgacaagctgtataagatgctcaccgaaatctacaccgaggataatttcgttaagttcttcaaggtcctgaaccggaagacctacctgaacttcgacaaggccgtgttcaagatcaacatcgtgcctaaagtgaactacaccatctacgacgggtttaacctgaggaacaccaacctggccgctaacttcaacgggcagaacacagagatcaacaacatgaatttcacgaagttgaagaacttcaccggactgtttgagttctacaaattgctgtgtgtgcgcgggatcatcactagcaagaccaagagccttgacaaaggctacaacaagtga

SEQ ID NO: 19 - DNA, 24 bp, synthetic construct
  source 1..24: mol_type = other DNA
ggagccgggaggttcgccactgac

SEQ ID NO: 20 - DNA, 24 bp, synthetic construct
  source 1..24: mol_type = other DNA
gtcagtggcgaacctcccggctcc

(19) United States
(12) Patent Application Publication
(10) Pub. No.: US 2025/0230199 A1
(43) Pub. Date: Jul. 17, 2025

(54) METHOD FOR INCREASING HALF-LIFE OF BOTULINUM TOXIN TYPE A LIGHT CHAIN PROTEIN

(71) Applicant: UBIPROTEIN. CORP, Seoul (KR)

(72) Inventors: Myung-Sun KIM, Wonju-si (KR); Hye-Young LIM, Seoul (KR); Su-Kyung OH, Yongin-si (KR); Jae-Woo LEE, Anyang-si (KR); Sung-Ryul BAE, Seongnam-si (KR); Seung-In CHOI, Seoul (KR)

(21) Appl. No.: 19/022,740

(22) Filed: Jan. 15, 2025

Related U.S. Application Data
(63) Continuation of application No. PCT/KR2023/010454, filed on Jul. 19, 2023.

(30) Foreign Application Priority Data
Jul. 19, 2022 (KR) 10-2022-0088602

(51) Int. Cl. C07K 14/33; A61K 39/00; C12N 15/70

(52) U.S. Cl. CPC C07K 14/33; C12N 15/70; A61K 39/00

(57) ABSTRACT

The present invention relates to: a method for increasing the half-life of a botulinum toxin type A light chain (AUT-BT-LC) protein, the method comprising substituting one or more lysine residues present in the amino acid sequence of the botulinum toxin type A light chain (BT-LC) protein; or a botulinum toxin type A light chain (AUT-BT-LC) protein having an increased half-life. The protein having an increased half-life according to the present invention may be used in a cosmetic composition and a pharmaceutical composition.

### CROSS-REFERENCE TO RELATED APPLICATIONS

This application is a Continuation of International Application No. PCT/KR2023/010454 filed Jul. 19, 2023, which claims priority from Korean Application No. 10-2022-0088602 filed Jul. 19, 2022. The aforementioned applications are incorporated herein by reference in their entireties.

### REFERENCE TO ELECTRONIC SEQUENCE LISTING

The application contains a Sequence Listing which has been submitted electronically in .XML format and is hereby incorporated by reference in its entirety. Said .XML copy, created on Jan. 15, 2025, is named “Seq_KOR-P30002C.xml” and is 29,209 bytes in size. The sequence listing contained in this .XML file is part of the specification and is hereby incorporated by reference herein in its entirety.

### TECHNICAL FIELD

The present invention relates to a method for increasing the half-life of botulinum toxin type A by substituting one or more amino acid residues in the light chain region of botulinum toxin type A. The method also relates to a botulinum toxin type A light chain protein with an increased half-life produced by the method.

### RELATED ART

In eukaryotic cells, 80-90% of proteins are degraded by the ubiquitin-proteasome pathway (UPP). Ubiquitin is a highly conserved protein composed of 76 amino acids that is present in almost all eukaryotic cells, of which 6th, 11th, 27th, 29th, 33rd, 48th, and 63rd are lysine (Lys, K), and residues 48 and 63 amino acid residues play a major role in forming polyubiquitin chains. The ubiquitin-proteasome pathway involves two separate sequential processes: the first process covalently tags substrates with multiple ubiquitin molecules, and the second process in which ubiquitin-tagged proteins are degraded by the 26S proteasome complex. Ubiquitin-substrate bond occurs via an isopeptide bond between a lysine residue of the substrate molecule and a glycine at the C-terminus of ubiquitin, and is achieved by the formation of a thiol ester between ubiquitin and the enzyme by ubiquitin-activating enzyme E1, ubiquitin-conjugating enzyme E2, and ubiquitin ligase E3. Of these, E1 (ubiquitin-activating enzyme) activates ubiquitin through an ATP-dependent reaction. E2 (ubiquitin-conjugating enzyme) accepts activated ubiquitin from E1 at a cysteine residue in the ubiquitin-conjugating domain and transfers it to the E3 ligase or directly to the substrate protein. The E3 enzyme also catalyzes a stable isopeptide bond between a lysine residue of a substrate protein and a glycine residue of ubiquitin. Another ubiquitin can be linked to the C-terminal lysine residue of ubiquitin bound to a substrate protein, and if this process is repeated and multiple ubiquitin molecules are linked to the substrate protein in a branched shape to form a polyubiquitin chain, the protein is recognized by the 26S proteasome and selectively degraded.

Botulinum toxin is a neurotoxin produced by Clostridium botulinum, a gram-positive anaerobic bacterium found in spoiled meat and canned foods. There are eight types of the eneurotoxins, and seven of them, A, B, C, D, E, F, and G, have been reported to cause nerve paralysis. The size of the complex is usually about 150 kDa due to the bond of the heavy and light chains, but it has been reported that the size of the complex can be up to 900 kDa depending on the type of neurotoxin due to the bond of complexes of non-toxin proteins in addition to the botulinum toxin protein. Botulinum toxin type A is known as the most lethal biological agent, and among the current uses of botulinum toxin, in the case of botulinum toxin injected into skin tissue, the duration of action is within 3 to 6 months, so the nerve paralysis effect is reduced or regular treatment is required, and there is also a limitation that the effect is reduced due to the formation of antibodies against botulinum toxin in vivo due to repeated administration.

In an effort to develop a botulinum toxin type A protein having an increased half-life, the inventors have identified that the half-life of a botulinum toxin type A light chain protein can be increased by substituting certain amino acid residues thereof with arginine, and have completed the invention.

### SUMMARY

The present invention aims to provide a method for increasing the half-life of a botulinum toxin type A light chain protein.

Also, the present invention aims to provide a botulinum toxin type A light chain protein having an increased half-life, wherein one or more lysine residues present in the amino acid sequence of the light chain region of botulinum toxin type A are substituted.

The present invention also aims to provide a cosmetic raw material and pharmaceutical composition comprising a botulinum toxin type A light chain protein having an increased half-life.

To accomplish the above objectives, the present invention provides a method for increasing the half-life of a botulinum toxin type A light chain protein, comprising substituting one or more lysine residues present in the amino acid sequence of the botulinum toxin type A light chain protein. Specifically, the present invention provides a method for increasing the half-life of a botulinum toxin type A light chain (BT-LC) protein, comprising substituting at least one of the lysines that bind the C-terminal glycine of ubiquitin with arginine in the amino acid sequence of said botulinum toxin type A light chain (BT-LC) protein. More specifically, said protein has the amino acid sequence of SEQ ID NO: 1, wherein at least one of the lysine residues at positions 89, 212, 301, 330, and 335 from the N-terminus thereof may be substituted with arginine.

The present invention also provides a botulinum toxin type A light chain (AUT-BT-LC) protein having an increased half-life, wherein at least one of the lysines that bind the C-terminal glycine of ubiquitin is substituted with arginine in the amino acid sequence of said protein. Specifically, said botulinum toxin type A light chain protein having an increased half-life has the amino acid sequence of SEQ ID NO: 1, wherein at least one of the lysine residues at positions 89, 212, 301, 330, and 335 from the N-terminus thereof may be substituted with arginine.

Also, the present invention provides a cosmetic raw material and/or cosmetic composition comprising said botulinum toxin type A light chain protein with an increased half-life.

Also, the present invention provides a pharmaceutical composition for the treatment of facial spasm, eyelid spasm, torticollis, blepharospasm, cervical dystonia, central pharyngeal dystonia, spasmodic dysphonia, migraine, anal pruritus, or hyperhidrosis, the pharmaceutical composition comprising said botulinum toxin type A light chain protein with an increased half-life and a pharmaceutically acceptable carrier or excipient.

In addition, to achieve said objectives, the present invention may provide an expression vector and/or a host cell comprising (a) a promoter; (b) a nucleic sequence encoding a botulinum toxin type A light chain (BT-LC) protein; and any linker.

In the present invention, a lysine residue of a botulinum toxin type A light chain protein may be substituted with a “conservative amino acid.” In the present invention, a “conservative amino acid” substitution means that an amino acid residue is substituted by another amino acid residue having a side chain with similar chemical properties, for example, charge or hydrophobicity. In general, conservative amino acid substitutions do not substantially change the functional properties of a protein. Examples of amino acid groups that have side chains with similar chemical properties comprise 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate; and 7) sulfur-containing side chains: cysteine and methionine.

In the present invention, lysine residues of a botulinum toxin type A light chain protein may be substituted by arginine or histidine contained in the basic side chain, preferably by arginine residues.

According to the present invention, a botulinum toxin type A light chain protein, in which one or more lysine residues present in the amino acid sequence of the botulinum toxin type A light chain protein are substituted with arginine, has an increased half-life and can remain in the body for a long period of time.

### BRIEF DESCRIPTION OF THE DRAWINGS

FIG. 1 shows the structure of a mammalian expression vector for a light chain region of botulinum toxin type A.

FIG. 2 illustrates an expression of a light chain region of botulinum toxin type A in HEK-293T cells. Expression was confirmed using a tagged Flag antibody.

FIG. 3 shows the ubiquitination of a light chain region of botulinum toxin type A by ubiquitination assay, suggesting that the degradation pathway of the light chain of botulinum toxin type A is a ubiquitin-proteasome pathway.

FIG. 4 shows the degree of ubiquitination in the light chain region of botulinum toxin type A in which lysine residues have been substituted with arginine, compared to a wild type.

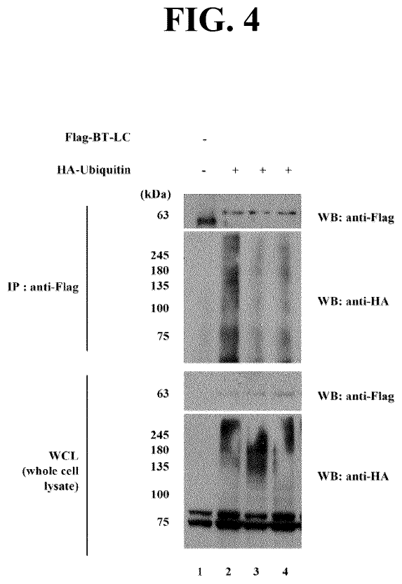

FIG. 5 shows the change in half-life of a light chain region of botulinum toxin type A after treatment with the protein synthesis inhibitor cycloheximide (CHX).

FIG. 6 is a graph of the change in half-life of a light chain region of botulinum toxin type A.

FIG. 7 shows the structure of an E. coli expression vector for a light chain region of botulinum toxin type A.

FIG. 8 illustrates the expression of a light chain region of botulinum toxin type A in E. coli, showing the purity per purification step and the purity of the final product, and also provides a numerical representation of the purification process for each step.

### DETAILED DESCRIPTION

In one embodiment of the present invention, the protein is a botulinum toxin type A light chain. In the amino acid sequence of the light chain region of botulinum toxin type A, represented by SEQ ID NO: 1, at least one of the 89th, 212th, 301st, 330th, and 335th lysine residues from the N-terminus is substituted with an arginine residue.

In another embodiment of the invention, the botulinum toxin type A light chain protein with an increased half-life obtained by said substitution can be used as a cosmetic raw material or for the preparation of pharmaceutical compositions for the treatment of facial spasm, eyelid spasm, torticollis, blepharospasm, cervical dystonia, central pharyngeal dystonia, spasmodic dysphonia, migraine, anal pruritus, or hyperhidrosis. (Long-term stable efficacy of botulinum toxin A in facial movement disorders with no need for increasing dose. S Badarny et al. Medicine (Baltimore) (2021 Jun. 25; 100(25):e26481; Botulinum Toxin for the Treatment of Hemifacial Spasm: An Update on Clinical Studies. Nicola Tambasco et al., 2021 Dec. 9; 13(12):881.)

Hereinafter, the invention will be described in more detail by way of example. The following examples are intended only to illustrate the present invention, and the present invention is not limited by the following examples.

### Example: Analysis of Ubiquitination of Botulinum Toxin Type A Light Chain (BT-LC) Protein and Preparation of Five Substituted Compounds with Increased Half-Life

### Example 1: Cloning into Mammalian Expression Vector and Confirmation of Protein Expression

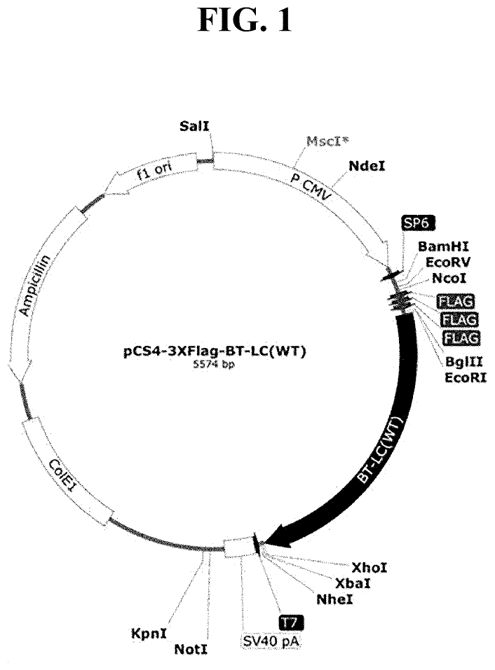

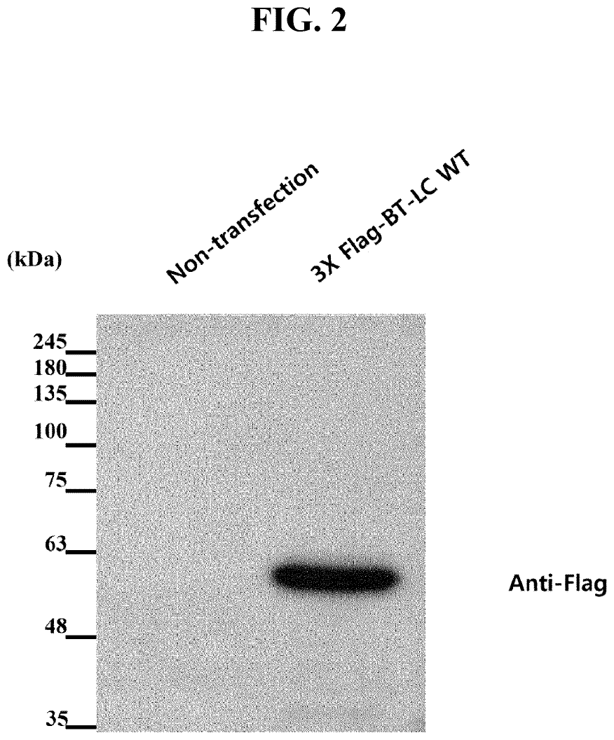

A light chain gene of botulinum toxin type A with SEQ ID NO: 1 was obtained by commissioning gene synthesis, and a fragment was made with EcoRI and XhoI for insertion into a mammalian expression vector, followed by conjugation and cloning. The structure of the above expression vector was schematized with SnapGene Viewer software and shown in FIG. 1 (in FIG. 1, the light chain amino acid sequence of botulinum toxin type A is as shown in SEQ ID NO: 1). To confirm that the constructed DNA in this way was properly expressed as a protein, the flag present in the pCS4-flag vector shown in the map in FIG. 1 was subjected to Western blotting using anti-flag (Sigma-aldrich, F1804) antibody. As a result, it was confirmed that the light chain protein of botulinum toxin type A bound to the flag was well expressed (FIG. 2).

### Example 2: In Vivo Ubiquitination Assay

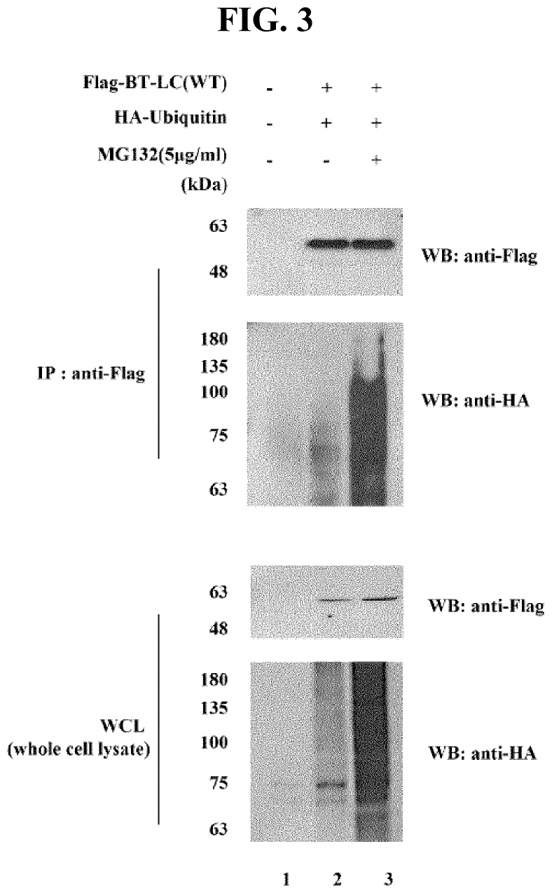

HEK-293T cells (source: Abcam) were transfected using plasmids encoding pCS4-flag-botulinum toxin type A light chain WT and pMT123-HA-Ubiquitin DNA (J Biol Chem., 279(4), 2368-2376, 2004; Cell Research, 22, 873-885, 2012; Oncogene, 22, 1273-1280, 2003; Cell, 78, 787-798, 1994). To confirm the extent of ubiquitination, 5 μg of pCS4-flag-botulinum toxin type A light chain WT and 1 μg of pMT123-HA-ubiquitin DNA were co-transfected into cells. 24 hours after transfection, and treated with MG132 (source: Sigma-Aldrich, proteasome inhibitor, 5 μg/ml) for 4 hours, followed by immunoprecipitation assay (FIG. 3).

Samples obtained for immunoprecipitation were lysed with lysis buffer (1% Triton X, 150 mM NaCl, 50 mM Tris-HCl, pH 8, and 1 mM PMSF (phenylmethanesulfonyl fluoride)), mixed with anti-flag (Sigma-aldrich, F1804) primary antibody and incubated overnight at 4° C. Immunoprecipitates were separated by reacting for 2 hours at 4° C. using protein A/G beads (Santa Cruz Biotechnology). This was followed by two washes with lysis buffer. For immunoblotting, protein samples were mixed with 2×SDS buffer, heated at 100° C. for 7 minutes, and separated by performing SDS-PAGE. The separated proteins were transferred to a polyvinylidene difluoride (PVDF) membrane, and then the membrane was developed by an ECL system (Western blot detection kit, ABfrontier, Seoul, Korea) using a blocking solution containing anti-Flag (Sigma-aldrich, F3165), anti-HA (Santa Cruz Biotechnology, sc-7392), and anti-3-actin (Santa Cruz Biotechnology, sc-47778) at a weight ratio of 1:1000 and an anti-mouse (Peroxidase-labeled antibody to mouse IgG (H+L), KPL, 074-1806) secondary antibody.

As a result, when immunoprecipitation was performed with anti-flag (Sigma-aldrich, F1804), ubiquitin was bound to the pCS4-flag-botulinum toxin type A light chain WT to form polyubiquitination, so that diffused ubiquitin was detected, resulting in darker bands (FIG. 3, lane 3).

### Example 3: Preparation of Substituted Compounds where Lysine (K) Residues were Substituted with Arginine

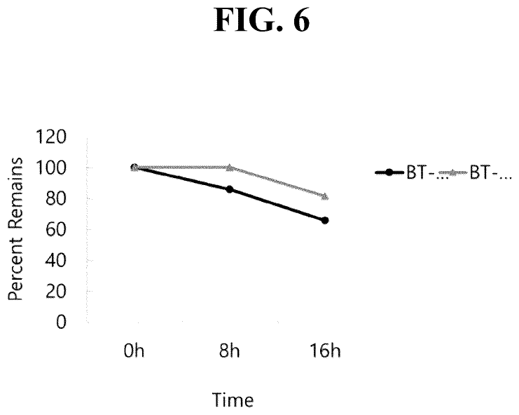

Lysine residues were substituted with arginine using site-directed mutagenesis, and a DNA sequence to induce a specific mutation was used to generate primers (BT-LC K89R FP 5′-GAT AAT TAT TTA AGG GGA GTT ACA AAA-3′ (SEQ ID NO:2), RP 5′-TTT TGT AAC TCC CCT TAA ATA ATT ATC-3′ (SEQ ID NO: 3); BT-LC K212R FP 5′-GGT GCA GGC AGA TTT GCT ACA GAT-3′ (SEQ ID NO: 4), RP 5′-ATC TGT AGC AAA TCT GCC TGC ACC-3′ (SEQ ID NO: 5); BT-LC K301R FP 5′-CTT AAT AAA GCT AGA TCA ATA GTA GGT-3′ (SEQ ID NO: 6), RP 5′-ACC TAC TAT TGA TCT AGC TTT ATT AAG-3′ (SEQ ID NO: 7); BT-LC K330R FP 5′-ACA TCT GGA AGA TTT TCG GTA GAT-3′ (SEQ ID NO: 8), RP 5′-ATC TAC CGA AAA TCT TCC AGA TGT-3′ (SEQ ID NO: 9); BT-LC K335R FP 5′-TTT TCG GTA GAT AGA TTA AAA TTT GAT-3′ (SEQ ID NO: 10), RP 5′-ATC AAA TTT TAA TCT ATC TAC CGA AAA-3′ (SEQ ID NO: 11)), and then PCR was performed under specific conditions to produce plasmid DNA where specific amino acid residues were substituted. pCS4-flag-botulinum toxin type A light chain (BT-LC) was used as a template, and plasmid DNA where lysine residues were substituted with arginine (K→R) was prepared (Table 1, FIG. 6 and light chain amino acid sequence of botulinum toxin type A: SEQ ID NOS: 12-16).

TABLE 1

| Lysine (K) | Botulinum toxin type A light chain (BT-LC) construct |
|---|---|
| Residue position | where lysine (K) is substituted with arginine (R) |
| 89 | pCS4-flag-botulinum toxin type A light chain (BT-LC) |
| | (K89R) |
| 212 | pCS4-flag-botulinum toxin type A light chain (BT-LC) |
| | (K212R) |
| 301 | pCS4-flag-botulinum toxin type A light chain (BT-LC) |
| | (K301R) |
| 330 | pCS4-flag-botulinum toxin type A light chain (BT-LC) |
| | (K330R) |
| 335 | pCS4-flag-botulinum toxin type A light chain (BT-LC) |
| | (K335R) |

Furthermore, the botulinum toxin type A light chain region (BT-LC) having a sequence that allows effective expression in E. coli by codon optimization was linked to pET21b and used as a template, and plasmid DNA was prepared in which lysine residues were substituted with arginine (K→R) (Table 2, FIG. 6, botulinum toxin A light chain amino acid sequence: SEQ ID NO: 17 and nucleotide sequence: SEQ ID NO: 18).

TABLE 2

| Lysine (K) | Botulinum toxin type A light chain (BT-LC) construct |
|---|---|
| Residue position | where lysine (K) is substituted with arginine (R) |
| 212 | pET21b-codon-optimized botulinum toxin type A light |
| | chain (BT-LC) (K212R) |

### Example 4. Confirmation of the Half-Life of Botulinum Toxin Type A Light Chain (BT-LC) by Protein Synthesis Inhibitor Cycloheximide (CHX)

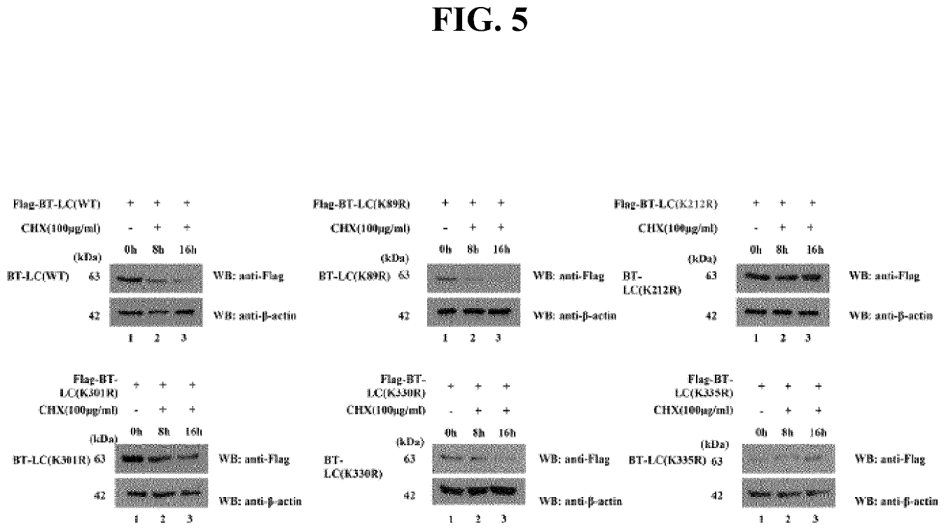

HEK 293T cells were respectively transfected with 2 g of pCS4-flag-botulinum toxin type A light chain (BT-LC) WT, pCS4-flag-botulinum toxin type A light chain (BT-LC) (K89R), pCS4-flag-botulinum toxin type A light chain (BT-LC) (K212R), pCS4-flag-botulinum toxin type A light chain (BT-LC) (K301R), pCS4-flag-botulinum toxin type A light chain (BT-LC) (K330R), pCS4-flag-botulinum toxin type A light chain (BT-LC) (K335R), and 48 hours later, were treated with the protein synthesis inhibitor cycloheximide (CHX) (Sigma-Aldrich) (100 g/ml), and the half-life was measured over 8 and 16 hours, and it was confirmed that the degradation of botulinum toxin type A light chain (BT-LC) was inhibited (FIG. 5). As a result, it was confirmed that the half-lives of the substituted botulinum toxin type A light chain (BT-LC) (K212R) were prolonged compared to the half-life of the botulinum toxin type A light chain (BT-LC), and these results were shown in a graph (FIG. 6).

### Example 5: Cloning into E. coli Expression Vector and Confirmation of Protein Expression

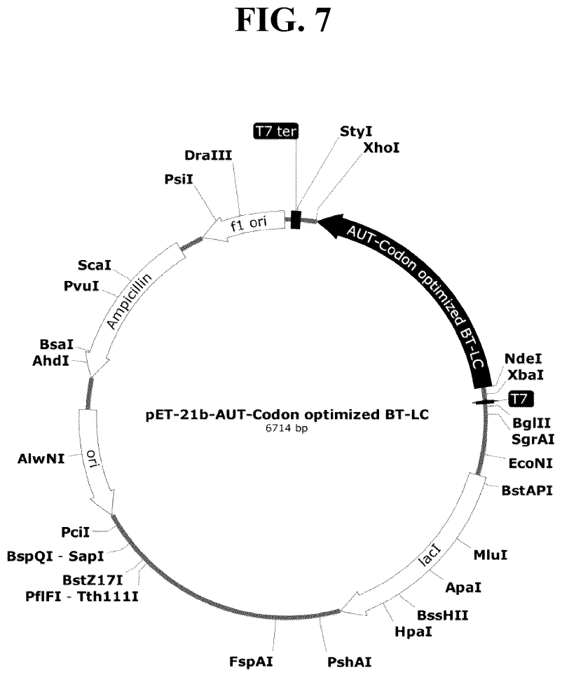

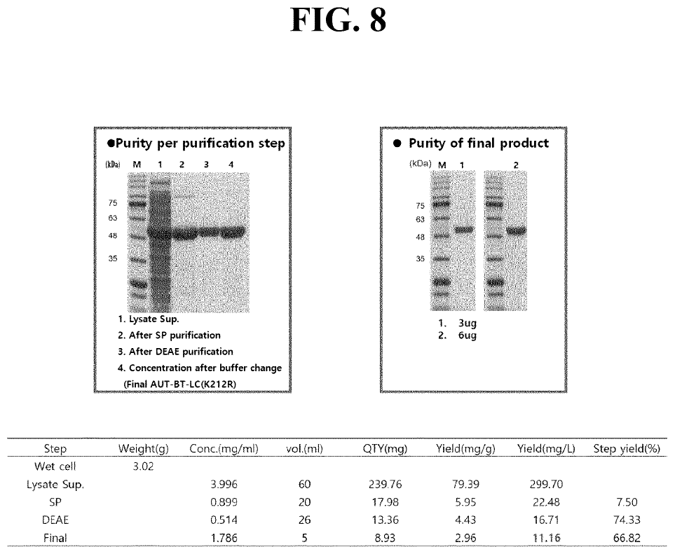

To insert the light chain region of botulinum toxin type A into the E. coli expression vector through the codon optimization process, a fragment was made from the pCS4-flag-codon-optimized botulinum toxin type A light chain DNA with NdeI and XhoI, and then conjugated with the pET21b vector and cloned. The inserted GCT sequence was removed using site-directed mutagenesis and diagrammed using SnapGene Viewer software (FIG. 7). To express the light chain region of botulinum toxin type A, it was treated with IPTG (1 mM) and expression was induced overnight at 18° C. Afterwards, coomassie staining was performed to confirm expression, and as a result, it was confirmed that a botulinum toxin type A light chain protein was well expressed (FIG. 8).

What is claimed is:

1. A method for increasing the half-life of a botulinum toxin type A light chain (BT-LC) protein, the method comprising substituting at least one of the lysines that bind the C-terminal glycine of ubiquitin with arginine in the amino acid sequence of said botulinum toxin type A light chain (BT-LC) protein.

2. The method of claim 1 wherein said protein has the amino acid sequence of SEQ ID NO: 1, and wherein at least one of the lysine residues at positions 89, 212, 301, 330, and 335 from the N-terminus thereof is substituted with arginine.

3. A botulinum toxin type A light chain (AUT-BT-LC) protein having an increased half-life, wherein at least one of the lysines that bind the C-terminal glycine of ubiquitin is substituted with arginine in the amino acid sequence of said protein.

4. The protein of claim 3, wherein said protein has the amino acid sequence of SEQ ID NO: 1, and wherein at least one of the lysine residues at positions 89, 212, 301, 330, and 335 from the N-terminus thereof is substituted with arginine.

5. Cosmetic raw material comprising the botulinum toxin type A light chain protein of claim 4 with an increased half-life.

6. A pharmaceutical composition for the treatment of facial spasm, eyelid spasm, torticollis, blepharospasm, cervical dystonia, central pharyngeal dystonia, spasmodic dysphonia, migraine, anal pruritus, or hyperhidrosis, the pharmaceutical composition comprising the botulinum toxin type A light chain protein of claim 4 with an increased half-life and a pharmaceutically acceptable carrier or excipient.

7. An expression vector comprising (a) a promoter; (b) a nucleic sequence encoding a botulinum toxin type A light chain (BT-LC) protein; and any linker, wherein said promoter and nucleic sequence are operatively linked.

8. A host cell comprising the expression vector of claim 7.
